(19) United States
(12) Patent Application Publication
(10) Pub. No.: US 2024/0150265 A1
(43) Pub. Date: May 9, 2024

(54) TERPENE EXTRACTION APPARATUS AND PROCESS

(71) Applicant: Tandem Technology Corporation, Portland, OR (US)

(72) Inventors: William KIMMERLE, Portland, OR (US); Kyle KIMMERLE, Portland, OR (US)

(21) Appl. No.: 18/503,883

(22) Filed: Nov. 7, 2023

Related U.S. Application Data
(60) Provisional application No. 63/382,811, filed on Nov. 8, 2022.

(51) Int. Cl. C07C 7/10

(52) U.S. Cl. CPC C07C 7/10

(57) ABSTRACT

A terpene extraction process may include the steps of: (A) providing a container with a biomass containing extractable materials including both water and terpenes; (B) passing a first gas over the biomass; (C) moving at least a portion of the extractable materials from the biomass into the first gas to form a second gas; (D) moving the second gas into a cold trap assembly to cool the second gas; (E) measuring a water vapor content of the second gas; (F) comparing the water vapor content to a predetermined value; and (G) stopping the extraction process when the water vapor content is less than the predetermined value.

This application claims priority to U.S. Provisional Patent Application No. 63/382,811, titled Terpene Extraction Apparatus And Process, filed Nov. 8, 2022, which is incorporated herein by reference.

### BACKGROUND

The present subject matter generally relates to apparatus and methods related to the extraction of terpenes. More specifically, the present subject matter relates to an apparatus related to the extraction of terpenes and a process for using and controlling same.

### SUMMARY

Provided is a terpene extraction process comprising providing a container with a biomass therein, the biomass containing a fixed amount of extractable materials, the extractable materials comprising both water and terpenes; passing a first gas mixture over the biomass; moving extractable materials from the biomass into the first gas mixture to form a second gas mixture of both the extractable materials and the first gas mixture; using a vacuum pump to move the second gas mixture into a condenser; measuring the water vapor content of the second gas mixture to generate a measurement of water vapor content; comparing the measurement of water vapor content of the second gas mixture to a predetermined critical value, and, where the measurement of water vapor content of the second gas mixture is less than the predetermined critical value, stopping the extraction process.

Further provided is a terpene extraction apparatus.

### BRIEF DESCRIPTION OF THE DRAWINGS

The present subject matter may take physical form in certain parts and arrangement of parts, embodiments of which will be described in detail in this specification and illustrated in the accompanying drawings which form a part hereof and wherein:

FIG. 1A is a section view of a first embodiment of a cold trap assembly taken along line 1A-1A of FIG. 1B.

FIG. 1B is a top view of the first embodiment of a cold trap assembly.

FIG. 1C is a first perspective view of the first embodiment of a cold trap assembly.

FIG. 1D is a second perspective view of the first embodiment of a cold trap assembly.

FIG. 2A is a top view of a first embodiment of a cold trap vessel.

FIG. 2B is a section view of the first embodiment of a cold trap vessel taken along line 2B-2B of FIG. 2A.

FIG. 2C is a front view of the first embodiment of a cold trap vessel.

FIG. 2D is a perspective view of the first embodiment of a cold trap vessel.

FIG. 3A is a top view of a first embodiment of a separation plate.

FIG. 3B is an end view of the first embodiment of a separation plate.

FIG. 3C is a perspective view of the first embodiment of a separation plate.

FIG. 4A is a top view of a first embodiment of a cold trap lid assembly.

FIG. 4B is a section view of the first embodiment of a cold trap lid assembly taken along line 4B-4B of FIG. 4A.

FIG. 4C is a side view of the first embodiment of a cold trap lid assembly.

FIG. 4D is a perspective view of the first embodiment of a cold trap lid assembly.

FIG. 5A is a top view of a first embodiment of a cold trap lid.

FIG. 5B is a section view of the first embodiment of a cold trap lid taken along line 5B-5B of FIG. 5A.

FIG. 5C is a perspective view of the first embodiment of a cold trap lid.

FIG. 6A is a top view of a first embodiment of a cooler assembly.

FIG. 6B is a section view of the first embodiment of a cooler assembly taken along line 6B-6B of FIG. 6A.

FIG. 6C is a side view of the first embodiment of a cooler assembly.

FIG. 6D is a perspective view of the first embodiment of a cooler assembly.

FIG. 7A is a top view of a first embodiment of a coolant chamber.

FIG. 7B is a section view of the first embodiment of a coolant chamber taken along line 7B-7B of FIG. 7A.

FIG. 7C is a side view of the first embodiment of a coolant chamber.

FIG. 7D is a perspective view of the first embodiment of a coolant chamber.

FIG. 8A is a top view of a first embodiment of an insulator jacket.

FIG. 8B is a section view of the first embodiment of an insulator jacket taken along line 8B-8B of FIG. 8A.

FIG. 8C is a front view of the first embodiment of an insulator jacket.

FIG. 8D is a perspective view of the first embodiment of an insulator jacket.

FIG. 9A is a section view of a second embodiment of a cold trap vessel.

FIG. 9B is a perspective view of the second embodiment of a cold trap vessel.

FIG. 10A is a top view of a second embodiment of a cooler assembly.

FIG. 10B is a section view of the second embodiment of a cooler assembly taken along line 10B-10B of FIG. 10A.

FIG. 10C a section view of the second embodiment of a cooler assembly taken along line 10C-10C of FIG. 10B.

FIG. 11A is a top view of a second embodiment of a cold trap lid assembly.

FIG. 11B is a section view of the second embodiment of a cold trap lid assembly taken along line 11B-11B of FIG. 11A.

FIG. 11C is a perspective view of the second embodiment of a cold trap lid assembly.

FIG. 12 is a perspective view of inlet tubing for the second embodiment of a cold trap lid assembly.

FIG. 13A is a top view of a first embodiment of a biomass container.

FIG. 13B is a section view of the first embodiment of a biomass container.

FIG. 13C is a perspective view of the first embodiment of a biomass container.

FIG. 14 is a block diagram of the interconnected components in one embodiment of an assembly adapted for use in terpene extraction process.

### DETAILED DESCRIPTION

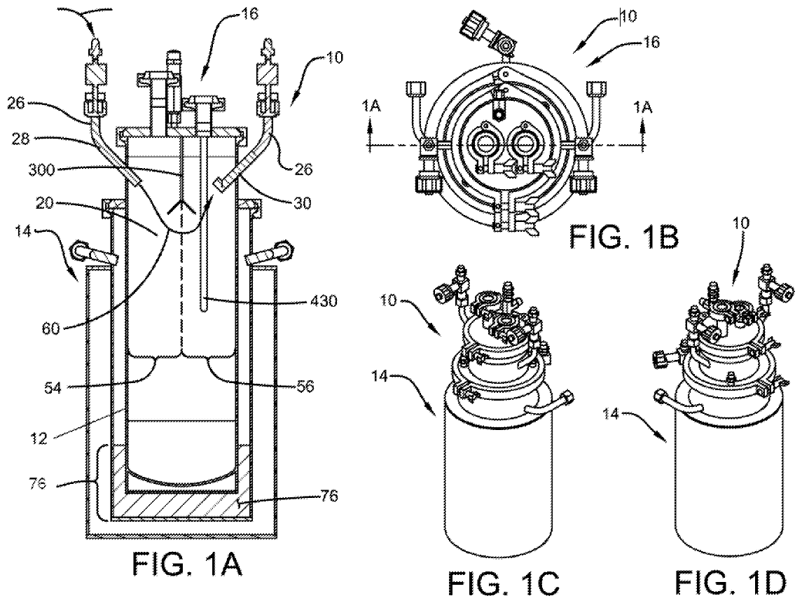

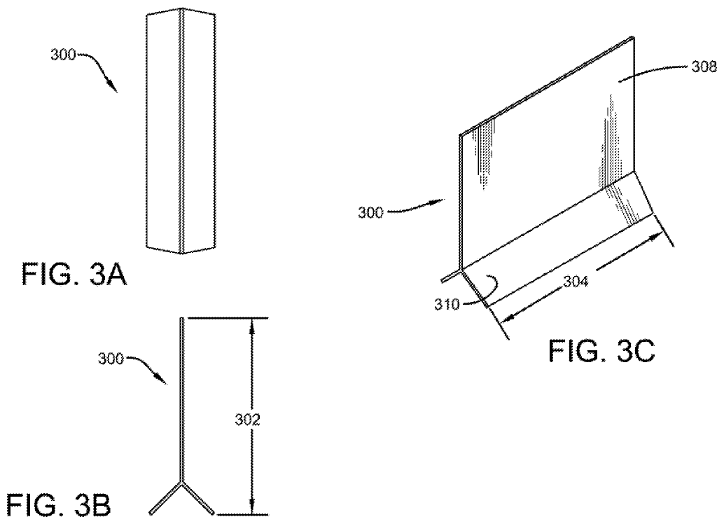

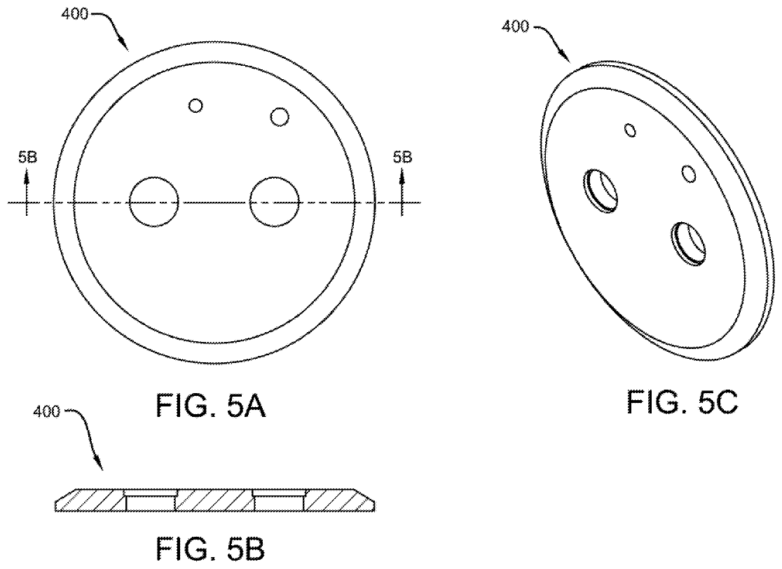

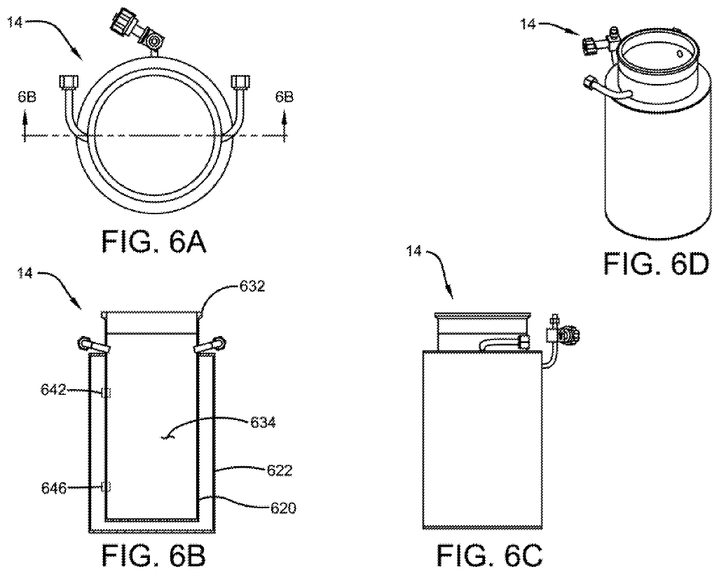

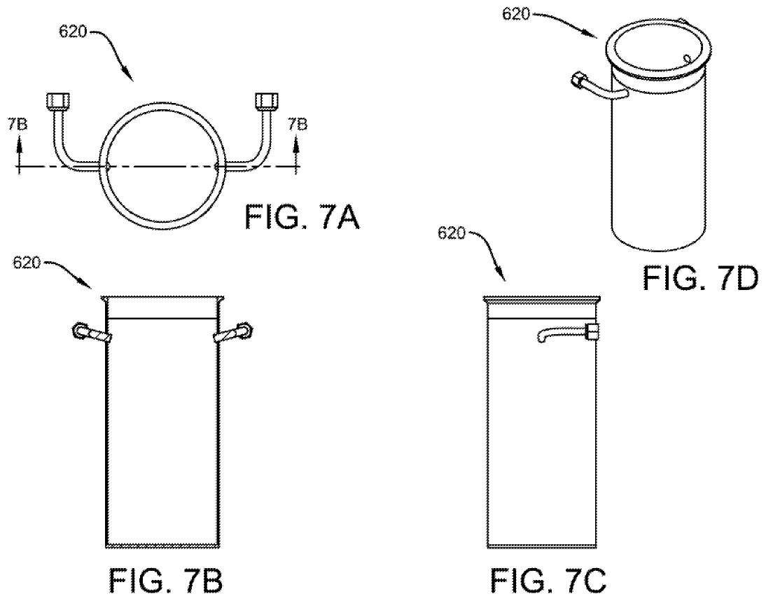

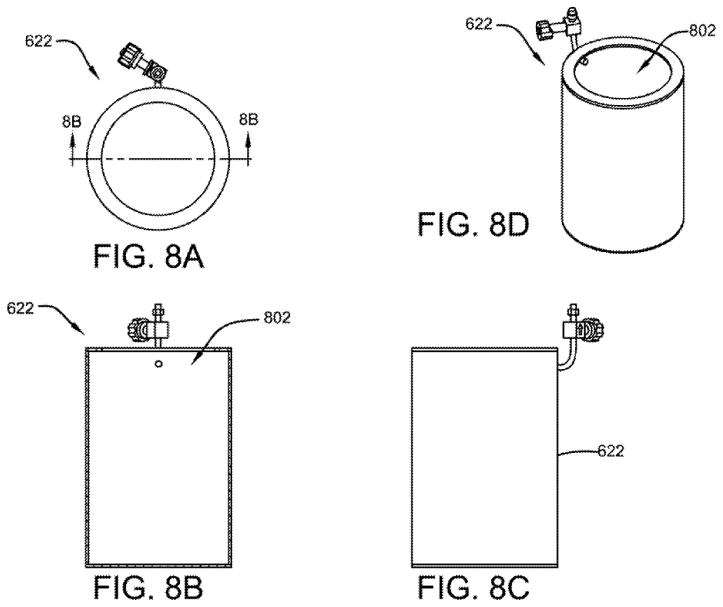

Referring now to the drawings wherein the showings are for purposes of illustrating embodiments of the present subject matter only and not for purposes of limiting the same, and wherein like reference numerals are understood to refer to like components, FIGS. 1-8 show a first non-limiting embodiment of a cold trap assembly 10. Cold trap assembly 10 may include a cold trap vessel 12 positioned within a cooler assembly 14 and a cold trap lid assembly 16 attached to the top of the cold trap vessel 12. As will be discussed hereinbelow, cold trap assembly 10 may be adapted for use in a terpene extraction process.

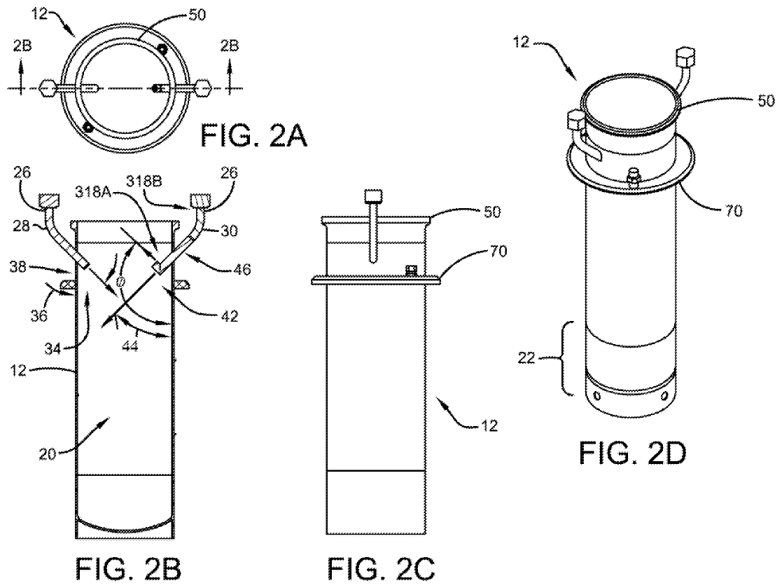

With reference now to FIGS. 2A-2D, the cold trap vessel 12 may include an elongated cylinder with a hollow interior 20 and a base 22, as shown. In other embodiments, the cold trap vessel 12 may be a hollow elongated prism or like structure. The base 22 may form a closure that may be welded onto the bottom end of the vessel 12 to seal off the bottom end from the surrounding environment. Alternatively, it is acceptable for the base 22 to be operationally engaged with the bottom end by bolting or other means chosen with good engineering judgment.

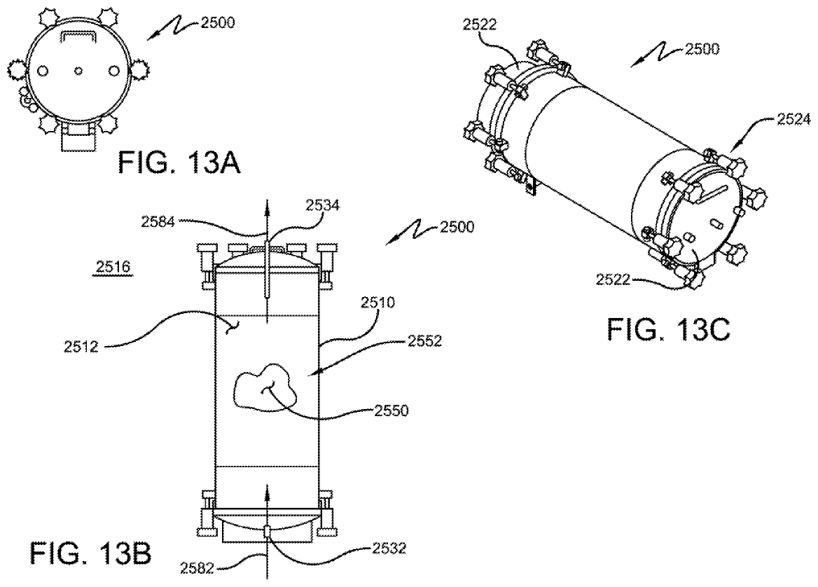

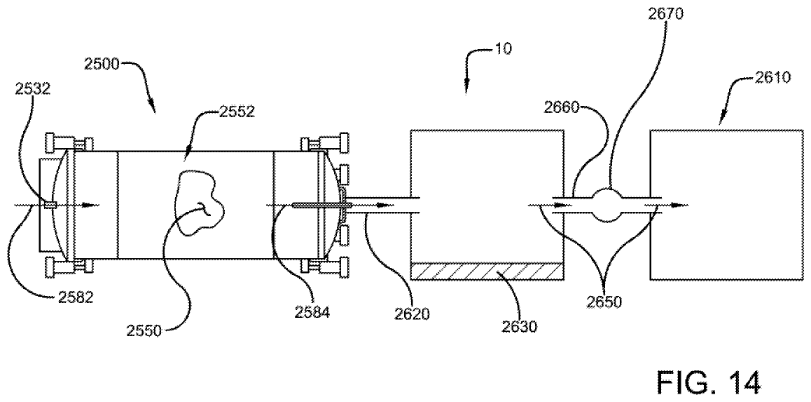

With reference now to FIGS. 1-2, a set of fluid communication lines 26 may comprise a cold trap inlet 28 and a cold trap outlet 30. The inlet 28 and the outlet 30 may each comprise a pipe, tube or similar elongated structure having first open end in fluid communication with a second open end, as shown. While the open ends exterior to the vessel 12 are shown capped in some of the drawings, FIGS. 1A-1D, this is not required and should not be considered limiting. The exterior open end of the inlet 28 may comprise threads or other engagement features chosen with good engineering judgment suitable to making sound operable connection with other fluid conveying components, such as, but not limited to a hose, pipe, tube, or similar fluid conduct adapted to carry fluid to inlet 28. As described below, in some non-limiting embodiments, the exterior open end of inlet 28 may be operationally engaged with a biomass container 2500 (FIGS. 13-14) in such a way that the inlet 28 may receive a fluid, such as a second gas mixture from the biomass container. The inlet 28 may be engaged with the vessel 12 by passing the inlet 28 partially through the wall of the vessel 12, such that one open end is exterior to the vessel 12 while the other open end is interior to the vessel 12, as shown. In some embodiments, there is a substantially fluid tight seal between the cold trap inlet 28 and the wall of the vessel. In some embodiments, a substantially fluid tight seal between the inlet 28 and the wall is created using a weld, but there are other acceptable means including soldering, brazing, press fitting, use of an adhesive, or combinations thereof.

With reference now to FIG. 2B, in some non-limiting embodiments, the interior open end of inlet 28 may extend through the vessel wall and into the interior 20 of the vessel 12 by some non-zero inlet protrusion length 34. The inlet protrusion length 34 may, in some embodiments, be between 0 cm and 2 cm. In various other embodiments, the inlet protrusion length 34 may be 0.25 cm, 0.5 cm, 0.75 cm, 1.0 cm, 1.25 cm, 1.5 cm, 1.75 cm, or greater. In some embodiments, the interior open end of inlet 28 may be substantially linear and extend through the vessel wall and into the interior 20 at some inlet protrusion angle 36 with respect to an imaginary plane tangent to the vessel wall at the inlet 28 point of entry 38. The inlet protrusion angle 36 in various embodiments may be 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 80 degrees, 90 degrees or some other angle between 5 and 175 degrees.

With reference now to FIGS. 1-2, the exterior opening of the outlet 30 may comprise threads or other engagement features chosen with good engineering judgment suitable to making sound operable connection with other fluid conveying components, such as, but not limited to a hose, pipe, tube, or similar fluid conduct adapted to carry fluid away from the exterior opening of the outlet 30. As described below, in some non-limiting embodiments, the exterior outlet opening may be operationally engaged with a vacuum pump 2610 (FIG. 14) in such a way that the vacuum pump will pull at least a partial vacuum on the exterior outlet opening and thereby induce flow of a fluid out of the exterior outlet opening and indirectly to induce flow of a fluid, such as a second gas mixture from the biomass container, into the interior 20 of the vessel 12. The outlet 30 may be engaged with the vessel 12 by passing the outlet 30 partially through the wall of the vessel 12, such that one open end is exterior to the vessel 12 while the other open end is interior to the vessel 12, as shown. In some embodiments, there is a substantially fluid tight seal between the cold trap outlet 30 and the wall of the vessel. In some embodiments, a substantially fluid tight seal between the outlet 30 and the wall is created using a weld, but there are other acceptable means including soldering, brazing, press fitting, use of an adhesive, or combinations thereof.

With reference now to FIG. 2B, in some non-limiting embodiments, the interior open end of outlet 30 may extend through the vessel wall and into the interior 20 of the vessel 12 by some non-zero outlet protrusion length 42. The outlet protrusion length 42 may, in some embodiments, be between 0 cm and 2 cm. In various other embodiments, the outlet protrusion length 42 may be 0.25 cm, 0.5 cm, 0.75 cm, 1.0 cm, 1.25 cm, 1.5 cm, 1.75 cm, or greater. In some embodiments, the interior open end of outlet 30 may be substantially linear and extend through the vessel wall and into the interior 20 at some outlet protrusion angle 44 with respect to an imaginary plane tangent to the vessel wall at the outlet 30 point of entry 46. The outlet protrusion angle 44 in various embodiments may be 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 80 degrees, 90 degrees or some other angle between 5 and 175 degrees.

With reference now to FIGS. 1-5, the cold trap lid assembly 16 may include a lid 400 that, in some embodiments, is operationally engaged with a clamp flange 50 connected to, or made integral with, the cold trap vessel 12. This engagement is shown in FIGS. 1A-1D and permits the user to readily and selectably engage or disengage the lid 400 from the clamp flange 50. The method of engagement can be any chosen with good engineering judgment. In some embodiments, this engagement of the lid 400 with the clamp flange 50 seals the lid 400 to the vessel 12 preventing from fluid from exiting the vessel 12 via the lid 400 vessel 12 interconnection.

With reference now to FIGS. 1A, 3A-3C, 4B and 4D, in some embodiments, the cold trap lid assembly 16 may include a separation plate 300. The separation plate 300 may be engaged to the lid 400 and may be adapted to serve as a baffle between the cold trap inlet 28 and the cold trap outlet 30, such that the separation plate 300 separates the top interior region into an inlet side 54 on the side of the plate 300 with the cold trap inlet 28 and an outlet side 56 on the side of the plate 300 with the cold trap outlet 30, as shown in FIG. 1A. The separation plate 300 may help to establish a flow path 60 between the inlet 28 and the outlet 30 that is longer than an imaginary straight line connecting the inlet 28 and the outlet 30. The separation plate 300 may have a plate height 302 and a separation plate width 304. In some non-limiting embodiments, the separation plate height 302 could be 11.5 cm+/−5 cm. In some non-limiting embodiments, the separation plate height 302 may be between 1.0 cm and 20.0 cm. In some other non-limiting embodiments, the separation plate height 302 may be 1.0 cm, 2.0 cm, 3.0 cm, 4.0 cm, 5.0 cm, 6.0 cm, 7.0 cm, 8.0 cm, 9.0 cm, 10.0 cm, 11.0 cm, 12.0 cm, 13.0 cm, 14.0 cm, 15.0 cm, 16.0 cm or greater. Additional details regarding flow path 60 is set forth in greater detail below. In some embodiments, the separation plate 300 may be composed of a flat plate 308 and a fin plate 310. It should be understood that these latter components are non-limiting and that non-flat plates or different shapes and geometries are also acceptable in forming the separation plate 300. The separation plate 300 may extend across the interior 20 by the separation plate width 304.

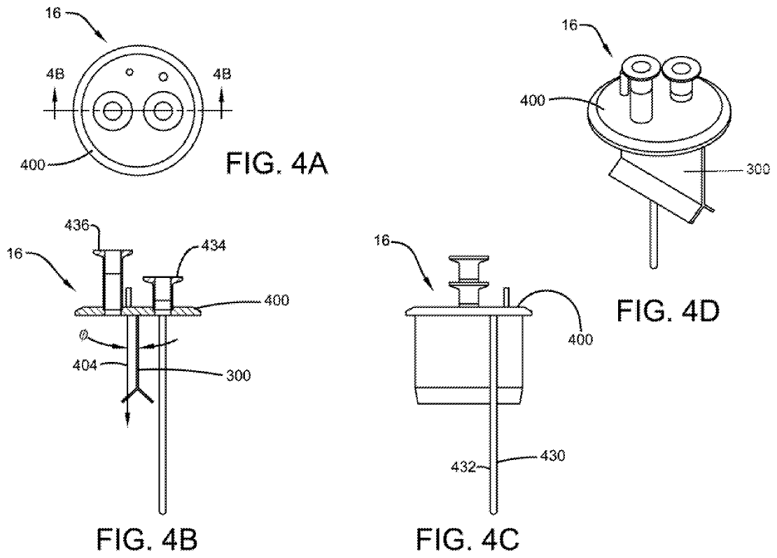

With continuing reference to FIGS. 1A, 3A-3C, 4B and 4D, in some embodiments, the separation plate 300 forms a sliding fit with the interior 20. It should be understood that different fit tolerances are acceptable: looser fit provides a lower bar to tolerancing standards and makes the separation plate 300 easier to operationally engage with the cold trap vessel 12; tighter fit provides fewer edge leakage sites for fluid to flow around the separation plate 300; in cases where the material for separation plate 300 is very elastic, it may be possible to establish acceptable operation with an interference fit between the separation plate 300 and the cold trap vessel 12. In some embodiments, the separation plate 300 extends into the interior region 20 by an amount that is a function of the separation plate height 302 and the angle of the separation plate .phi., see FIG. 4B, with an imaginary vector 404 normal to the cold trap lid 400. It should be understood that .phi. is shown as zero degrees in FIG. 4B, but that .phi. can range from 0 to 45 degrees in either direction. In some embodiments, the separation plate 300 is angled toward cold trap inlet 28 by some angle .phi. that is non-zero and less than 30 degrees. In some embodiments, .phi. is 30 degrees and the separation plate 300 is angled toward cold trap inlet 28. In some embodiments, .phi. is 25 degrees and the separation plate 300 is angled toward cold trap inlet 28. In some embodiments, .phi. is 20 degrees and the separation plate 300 is angled toward cold trap inlet 28. In some embodiments, .phi. is 15 degrees and the separation plate 300 is angled toward cold trap inlet 28. In some embodiments, .phi. is 10 degrees and the separation plate 300 is angled toward cold trap inlet 28. In some embodiments, .phi. is 5 degrees and the separation plate 300 is angled toward cold trap inlet 28. In some embodiments, the separation plate 300 is angled toward cold trap outlet 30 by some angle .phi. that is non-zero and less than 30 degrees. In some embodiments, .phi. is 30 degrees and the separation plate 300 is angled toward cold trap outlet 30. In some embodiments, .phi. is 25 degrees and the separation plate 300 is angled toward cold trap outlet 30. In some embodiments, .phi. is 20 degrees and the separation plate 300 is angled toward cold trap outlet 30. In some embodiments, .phi. is 15 degrees and the separation plate 300 is angled toward cold trap outlet 30. In some embodiments, .phi. is 10 degrees and the separation plate 300 is angled toward cold trap outlet 30. In some embodiments, .phi. is 5 degrees and the separation plate 300 is angled toward cold trap outlet 30. The angle .phi. of the separation plate 300 may be useful in determining how the flow velocity of the fluid changes as it flows through the interior 20.

With reference now to FIGS. 1A and 4A-4D, the cold trap lid assembly 16 may include a thermowell 430. The thermowell 430 may be adapted to extend into the interior 20 when the cold trap lid 400 is operationally engaged with the cold trap vessel 12. When so engaged as to extend into the interior 20, the thermowell 430 may be adapted to provide a region adapted for operational engagement of one or more transducers adapted to provide temperature data representative of the temperature of the interior 20. FIG. 1A, shows one non-limiting embodiment wherein the lid 400 is operatively engaged with the vessel 12 and the thermowell 430 extends within the interior 20. A thermometer, temperature probe, or other temperature sensor adapted to provide temperature data may be inserted into the thermowell 430. The thermowell 430 will typically be a thin wall 432 of material that defines a narrow cavity. The material of the thin wall 432 will conduct heat well enough that the temperature sensed by a temperature sensor within the thermowell 430 is representative of the temperature within the interior 20 proximate to the thermowell 430. In some non-limiting embodiments, the material of the thin wall 432 will be stainless steel, another steel, brass, bronze, copper, copper alloys, aluminum, aluminum alloys, silver, silver alloys, or some combination thereof. In some non-limiting embodiments, the thermowell 430 may be adapted to accommodate a temperature sensor, a heat sink, a heating element, or a combination thereof. In some non-limiting embodiments, the thermowell 430 as assembled may include a temperature sensor, a heat sink, and a heating element. Each of the latter temperature sensor, a heat sink, and a heating element may be conventional items of each sort. The thermowell 430 may be accessible through a thermowell port 434 in the lid 400. The cold trap lid 400 may optionally include an auxiliary port 436 which provides selectable communication through the lid 400.

With reference now to FIGS. 1-2, and 6-8, the cooler assembly 14 may have a cooler flange 632 to be operationally engaged with a connection collar 70 connected to, or made integral with, the cold trap vessel 12. The cooler assembly 14 may include a container 620 positioned within an insulation jacket 622. Container 620 may define a cooler interior 634 adapted to receive the cold trap vessel 12, to cool the cold trap vessel 12, and thereby to cool the contents of the cold trap vessel 12. In some embodiments when the cold trap vessel 12 is operationally engaged with the cooler assembly 14, the cooler assembly 14 at least partially encompasses the vessel 12. As shown in FIGS. 1A-1D, in some embodiments, when the cold trap vessel 12 is operationally engaged with the cooler assembly 14, the cold trap vessel 12 extends partway into the cooler assembly 14 with clearance beneath and around the cold trap vessel 12 and also extends from the cooler assembly 14 such that part of the cold trap vessel 12 is exposed to the surrounding environment. It is also contemplated that in some non-limiting embodiments, the cold trap vessel 12 will be entirely within the cooler assembly 14. As shown in FIGS. 1A-1D, operational engagement of the connection collar 70 with the cooler flange 632 holds the cold trap vessel 12 in position and orientation with respect to the cooler assembly 14. Operational engagement of the connection collar 946 with the cooler flange 632 may also close, at least partially, the cooler assembly 14 to insulate the cooler interior 634 from the surrounding environment. In some non-limiting embodiments, the connection collar 70 is ring-shaped and removably engageable with the cooler flange 632 to cover the cooler interior 634 while providing a fit through the ring opening for the cold trap vessel 12.

With continuing reference to FIGS. 1-2, and 6-8, the insulation jacket 622 may define an insulation jacket interior region 802 that is substantially evacuated to increase resistance to conductive and convective heat transfer. The insulation jacket 622 may be adapted to promote heat insulation of the cooler assembly 14 from the surrounding environment. In some embodiments, the insulation jacket 622 will surround or mostly surround the cooler assembly 14. In some embodiments, a small part of the cooler assembly 14 will extend outside of the insulation jacket 622 in order to facilitate service, operational connections, and other operation and maintenance. In some embodiments, the cooler assembly 14 is adapted to cool the cold trap vessel 12 to temperatures between −40 C and −197 C. In some embodiments, cooling the vessel 12 may entail operationally engaging the vessel 12 with the cooler assembly 14, and at least partially filling the cooler interior 634 with a coolant fluid 76. In some non-limiting embodiments coolant fluid 76 may be liquid nitrogen, liquid oxygen, liquid argon, liquid hydrogen, liquid helium, liquid methane, liquid ammonia, methanol, ethanol, isopropyl alcohol, some combination thereof, or other coolants chosen with good engineering judgment. In some embodiments at least partially filling the cooler interior 634 with a coolant fluid 76 will bring the coolant fluid 76 into contact with an exterior region of the cold trap vessel 12 and cool the vessel 12 and any contents therein by pulling heat from the vessel 12 into the coolant fluid 76. In some embodiments, the cooler assembly 14 may comprise coolant fluid level detector comprising a set of temperature sensors 642, 646 operationally engaged therewith, the set of temperature sensors 642, 646 comprising a first temperature sensor 646 at a first height and a second temperature sensor 642 at a second height different from the first height. The set of temperature sensors 642, 646 may be adapted to provide data about the level 78 of the coolant fluid 76 within the cooler interior 634. This latter data may take the form of an estimation of the level 78 of the coolant fluid 76. The level 78 of the coolant fluid 76 may be estimated to be between the first height and the second height where the first temperature sensor 646 shows a temperature consistent with the coolant level being at or above the first height and the second temperature sensor 642 shows a temperature consistent with the coolant level not being at or above the second height.

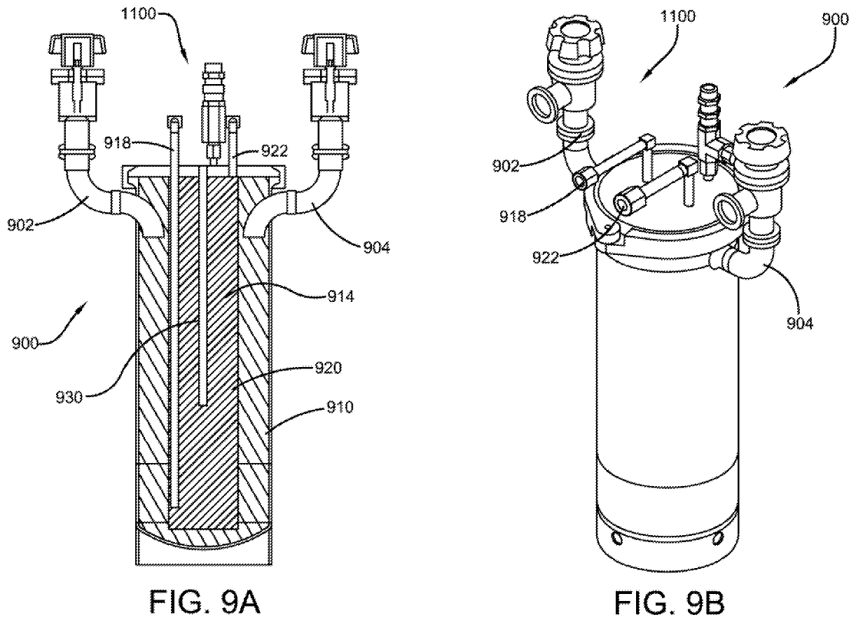

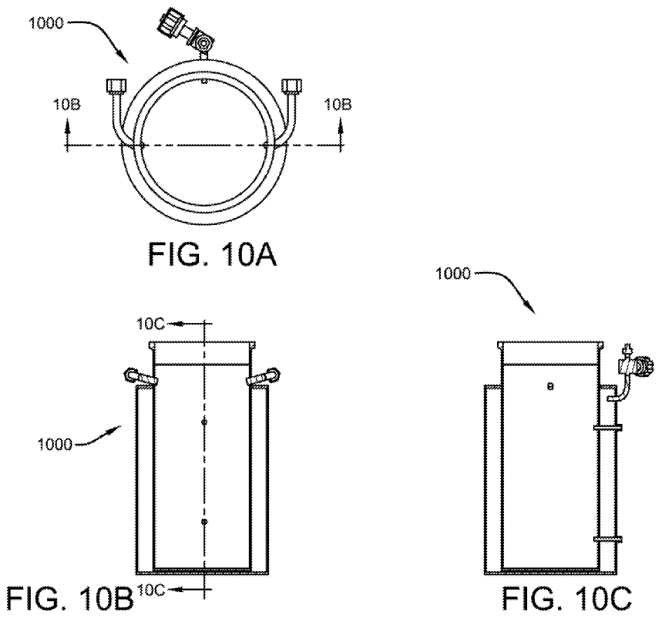

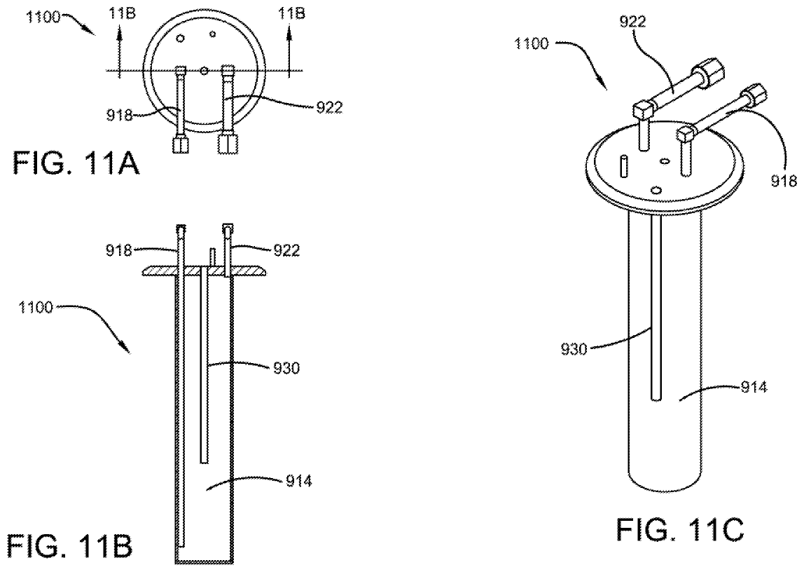

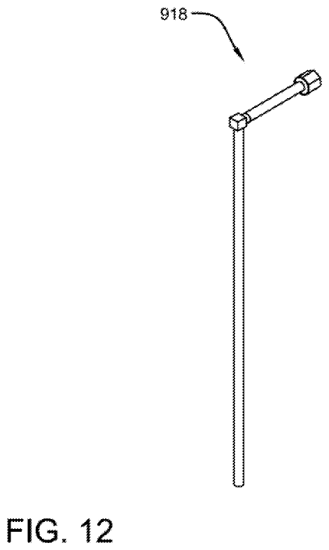

With reference now to FIGS. 9-12, a cold trap assembly incorporating different embodiments will now be described. Many of the components in this cold trap assembly work like the similar components described above. Therefore, the focus here will be on the differences. This cold trap assembly may include a cold trap vessel 900 that may be positioned within a cooler assembly 1000 and a cold trap lid assembly 1100 may be attached to the top of the cold trap vessel 900, as shown. This cold trap assembly may be adapted for use in a terpene extraction process. The vessel 900 may have an inlet 902 that receives the fluid 910 to be cooled and an outlet 904 where the cooled fluid or vapor exits the vessel 900. When the cold trap vessel 900 is positioned within the cooler assembly 1000, the cooler assembly 1000 may assist in cooling the fluid 910 like the cooler assembly 14 described above. For these embodiments, however, the fluid 910 is not just cooled via the exterior surface of the vessel 900 but also via an interior surface. Specifically, the cold trap lid assembly 1100 may include a coolant volume 914 that extends within the vessel 900 and may have an inlet 918 that receives a coolant 920 and an outlet 922 where the coolant exits the volume 914. For the embodiments shown, the inlet 918 includes piping or tubbing that extends near the bottom of the coolant volume 914 and the outlet 922 only extends slightly into the coolant volume 914, as shown. A thermowell 930 may be used to measure and control the temperature within the coolant volume 914 similar to the operation of the thermowell described above.

With reference now to FIGS. 13-14, shown is one non-limiting embodiment of a biomass container 2500. Biomass container 2500 may comprise a housing 2510 which defines an interior region 2512 and an exterior region 2516 and may have one or more selectably closable doors 2522 which permit the communication of biomass between the interior region 2512 and an exterior region 2516 when open. The doors 2522 may be closable using a set of one or more discrete fasteners 2524 as shown but other methods, such as a collar, or other means chosen with good engineering judgment are also contemplated. The interior region 2512 may be usable to contain a biomass therein which may be isolated from the exterior region 2516 by sealing the one or more selectably closable doors 2522. In some embodiments, housing 2510 may further comprise a set of one or more inlet apertures 2532 adapted for communication of a fluid such as a first gas mixture 2582 from the exterior region 2516 to the interior region 2512. In some embodiments, housing 2510 may further comprise a set of one or more outlet apertures 2534 adapted for communication of a fluid such as a second gas mixture 2584 from the interior region 2512 to the exterior region 2516. In some embodiments the housing 2510 may comprise both a set of one or more inlet apertures 2532, adapted for communication of a first gas mixture 2582 from the exterior region 2516 to the interior region 2512, and a set of one or more outlet apertures 2534 adapted for communication of a second gas mixture 2584 from the interior region 2512 to the exterior region 2516.

With continuing reference to FIGS. 13-14, in embodiments in which the housing 2510 comprises both a set of one or more inlet apertures 2532 and a set of one or more outlet apertures 2534, a terpene extraction process may be performed which includes, at least in part, providing a biomass container 2500 with a biomass 2550 within the interior region 2512 thereof, the biomass 2550 containing a fixed amount of extractable materials 2552 comprising both water and terpenes; passing a fluid flow, such as first gas mixture 2582, over the biomass 2550 by flowing a gas mixture, such as first gas mixture 2582, through the set of one or more inlet apertures 2532 into the interior region 2512 and over the biomass 2550; and moving extractable materials 2552 from the biomass 2550 into the gas mixture, such as first gas mixture 2582, to form a second gas mixture 2584 of both the extractable materials 2552 and the first gas mixture 2582. The movement of the extractable materials 2552 from the biomass 2550 into the first gas mixture 2582 to form a second gas mixture 2584 may be through diffusion, but any mechanism for the movement chosen with sound engineering judgement is acceptable. The second gas mixture 2854 may then flow through the set of one or more outlet apertures 2534 out of the interior region 2512. The one or more of the outlet apertures 2534 may be engaged with a pipe, tube, or other conduit such as, and without limitation, cold trap inlet 28 (FIG. 1A) or cold trap inlet 902 (FIG. 9A), adapted to convey the second gas mixture 2584 to one or more other components, such as, and without limitation, cold trap assembly 100, the cold trap assembly shown in FIGS. 9-12, another condenser, or other regions for subsequent handling or treatment. The movement of the second gas mixture out of the biomass container and into a cold trap assembly or other condenser may be performed using a pump, blower, fan, compressor, or vacuum pump 2610.

Still referring to FIGS. 13-14, in some non-limiting embodiments, a cold trap assembly may be in fluid communication with a vacuum pump 2610 through a cold trap outlet 30 (FIG. 1A) or a cold trap outlet 904 (FIG. 9A) such that the vacuum pump 2610 may be operated to pull at least a partial vacuum on the outlet and thereby induce flow of a fluid through the biomass container 2500 and then through the cold trap assembly as shown in FIG. 26. In the non-limiting embodiment shown, a first gas mixture 2582 flows into biomass container 2500 through an inlet aperture 2532, over biomass 2550 where it intermixes with extractable materials 2552 comprising a terpene and water to form second gas mixture 2584. Second gas mixture 2584 flows out of the biomass container 2500 through outlet aperture 2534 through a first conduit 2620 to cold trap assembly where at least some of the extractable materials 2552 condense out of the second gas mixture 2584 as a precipitate 2630. The remainder mixture 2650 from second gas mixture 2584 is pulled from the cold trap assembly and into the vacuum pump through a second conduit 2660.

With continuing reference to FIGS. 13-14, in some embodiments it may be desirable to heat or cool the biomass container 2500 during a terpene extraction process in order to establish a temperature within the biomass container 2500 that is high enough to promote diffusion of extractable materials 2552 from the biomass 2550 into the first gas mixture 2582 while still being cool enough to promote dissolution of the extractable materials 2552 into the first gas mixture 2582 to form the second gas mixture 2584. As noted above, in some embodiments, the extractable materials from the biomass moved into the first gas mixture to form a second gas mixture will comprise some amount of the water and terpenes from the biomass. It has been found that the amount of water in the extractable materials from the biomass moved into the first gas mixture to form a second gas mixture deceases as the process proceeds.

With reference now to FIG. 14, in some embodiments of a terpene extraction process, the process further comprises measuring the water vapor content of the second gas mixture to generate a measurement of water vapor content. In some embodiments measuring the water vapor content of the second gas mixture may be done using a hygrometer 2670. In some embodiments measuring the water vapor content of the second gas mixture may be done using a capacitive hygrometer, or a resistive hygrometer, or a thermal hygrometer, or a gravimetric hygrometer, or an optical hygrometer, or some combination thereof. In some embodiments of a terpene extraction process, the process further comprises comparing the measurement of water vapor content of the second gas mixture to a predetermined critical value, and, where the measurement of water vapor content of the second gas mixture is less than the predetermined critical value, stopping the extraction process.

With continuing reference to FIG. 14, the first gas mixture 2582 may comprise nitrogen, carbon dioxide, another gas or mixture of the aforementioned gases chosen with good engineering judgment. In the contemplated process, the first gas mixture will act as a solute gas in which the terpenes and water of the biomass will be dissolved to form the second gas mixture 2584. The biomass 2550 may be a primarily plant mass. The biomass 2550 may be one or more plants from the Cannabaceae family of plants which is described further hereinbelow. In some embodiments the biomass comprises plants from the genus Cannabis or the genus Humulus, or both. In some embodiments the biomass 2550 comprises Humulus lupulus, Cannabis sativa, Cannabis indica, or a mixture thereof. In some embodiments the biomass 2550 comprises Cannabis sativa, Cannabis indica, or a mixture thereof.

With reference to all the FIGURES, a method for terpene extraction may comprise: providing a container with a biomass therein, wherein the biomass may contain a fixed amount of extractable materials, wherein the extractable materials may comprise both water and terpenes; passing a first gas mixture over the biomass; moving extractable materials from the biomass into the first gas mixture to form a second gas mixture of both the extractable materials and the first gas mixture; moving the second gas mixture into a condenser; measuring the water vapor content of the second gas mixture to generate a measurement of water vapor content; and comparing the measurement of water vapor content of the second gas mixture to a predetermined critical value, and, where the measurement of water vapor content of the second gas mixture is less than the predetermined critical value, stopping the terpene extraction.

With reference to all the FIGURES, a method for extracting terpenes may comprise providing a terpene extraction cold trap assembly having a cold trap vessel with an interior region and an exterior region; a separation plate separating a top interior region into an inlet side and an outlet side, a cold trap inlet protruding into the interior region at an inlet point of entry into the vessel by an inlet protrusion length greater than 0.5 cm and forming an inlet protrusion angle with the vessel between 5 degrees and 175 degrees, a cold trap outlet protruding into the interior region at an outlet point of entry through the vessel by an outlet protrusion length greater than 0.5 cm, and forming an outlet protrusion angle with the vessel between 5 degrees and 175 degrees, and a thermowell extending into the interior region, the thermowell having therein a first temperature sensor adapted to provide temperature data representative of the temperature of the interior region; directing a flow of a gaseous solution or gaseous mixture comprising a mixture of a solute gas and a dissolved terpene therein into the inlet; directing the flow of the gaseous mixture into the vessel and cooling the gaseous mixture sufficiently to cause at least some of the dissolved terpene therein to precipitate from the solution as a liquid or solid; and collecting the precipitated liquid or solid terpene in the vessel. The solute gas may be nitrogen, carbon dioxide, or another gas or mixture of gases chosen with good engineering judgment. The dissolved terpene may be derived from one or more plants from the Cannabaceae family of plants. The Cannabaceae family of plants comprises the genus Cannabis and the genus Humulus. Hops, marijuana, and hemp are each plants from of the Cannabaceae family of plants from which one or more terpenes of interest may be extracted and turned into the above mentioned dissolved terpene. In some non-limiting embodiments, the dissolved terpene is derived from or extracted from Humulus lupulus. In some non-limiting embodiments, the dissolved terpene is derived from or extracted from Cannabis sativa. In some non-limiting embodiments, the dissolved terpene is derived from or extracted from Cannabis indica.

A method for extracting terpenes may further comprise cooling the vessel by operationally engaging the vessel with a cooler assembly and at least partially filling the cooler assembly with a coolant. In some non-limiting embodiment, the coolant may be liquid nitrogen but as noted above, other coolants are also acceptable. In some non-limiting embodiments in which both the coolant and the solute gas are nitrogen, the coolant and the solute gas may both be sourced from the same nitrogen reservoir.

A method may further comprise pulling the gaseous mixture into the cold trap inlet; pulling the gaseous mixture along flow path between the cold trap inlet and the cold trap outlet such that gaseous mixture is cooled by passing through the cooled inlet side; pulling the gaseous mixture around the separation plate; and pulling the gaseous mixture through the cooled outlet side. Cooling the gaseous mixture as recited above may cause the dissolved terpene to precipitate from the mixture as a liquid or solid. In those situations in which the dissolved terpene precipitates from the mixture while within the vessel, the precipitated liquid or solid terpene may collect proximate to a bottom end of the vessel and be collected therefrom. In some embodiments, terpene liquid or solid precipitate is permitted to accumulate in the vessel and is occasionally removed by disconnecting the associated components from the vessel and dumping it out. Alternatively, it may be acceptable to include a drop tube passing through the lid which may be used to draw off terpene liquid or solid precipitate.

In some non-limiting embodiments, the vessel may further comprise one or more heat exchange passages or tubes therethrough adapted to provide additional heat exchange surfaces and thereby to facilitate cooling of the vessel and contents thereof.

Numerous embodiments have been described herein. It will be apparent to those skilled in the art that the above methods and apparatuses may incorporate changes and modifications without departing from the general scope of the present subject matter. It is intended to include all such modifications and alterations in so far as they come within the scope of the appended claims or the equivalents thereof. Further, the “invention” as that term is used in this document is what is claimed in the claims of this document. The right to claim elements and/or sub-combinations that are disclosed herein as other inventions in other patent documents is hereby unconditionally reserved.

Having thus described the present subject matter, it is now claimed:

1-7. (canceled)

8. A terpene extraction process comprising the steps of:
(A) providing a container with a biomass containing extractable materials including both water and terpenes;
(B) passing a first gas over the biomass;
(C) moving at least a portion of the extractable materials from the biomass into the first gas to form a second gas;
(D) moving the second gas into a cold trap assembly to cool the second gas;
(E) measuring a water vapor content of the second gas;
(F) comparing the water vapor content to a predetermined value; and
(G) stopping the extraction process when the water vapor content is less than the predetermined value.

9. The terpene extraction process of claim 8 further comprising the step of:
decreasing water vapor content of the second gas as the terpene extraction process proceeds.

10. The terpene extraction process of claim 8 wherein step (D) comprises the step of:
causing at least some dissolved terpene in the second gas to precipitate as a liquid or solid.

11. The terpene extraction process of claim 8 wherein the biomass is primarily plant mass.

12. The terpene extraction process of claim 11 wherein the biomass comprises one or more plants from the Cannabaceae family of plants.

13. The terpene extraction process of claim 8 wherein step (B) comprises the step of:
passing nitrogen gas over the biomass.

14. The terpene extraction process of claim 8 wherein step (D) comprises the step of:
cooling the second gas with liquid nitrogen.

15. The terpene extraction process of claim 8 wherein step (D) comprises the step of:
providing the cold trap assembly with a second gas flow path between an inlet and an outlet;
providing the flow path between the inlet and the outlet with an interior surface and an exterior surface; and
providing coolant to cool the second gas via the interior surface and the exterior surface.

16. The terpene extraction process of claim 15 wherein:
a first coolant cools the second gas via the interior surface; and
a second coolant cools the second gas via the exterior surface.

17. The terpene extraction process of claim 16 wherein step (D) comprises the steps of:
providing a first thermowell adapted to provide temperature data representative of the interior surface; and
providing a second thermowell adapted to provide temperature data representative of the exterior surface.

18. The terpene extraction process of claim 8 wherein step (E) comprises the step of:
using a hygrometer to measure the vapor content of the second gas.

19. A terpene extraction apparatus comprising:
a biomass container having:
an inlet;
an outlet;
an interior having a biomass containing extractable materials including both water and terpenes;
a cold trap assembly having:
an inlet;
an outlet;
an interior with a coolant;
a gas moving device;
a water vapor content measuring device;
wherein the terpene extraction apparatus is designed to:
move a first gas into the inlet of the container;
move the first gas over the biomass in the interior of the container to form a second gas including at least a portion of the extractable materials;
move the second gas into the inlet of the cold trap assembly;
transfer heat from the second gas into the coolant to cool the second gas;
measure a water vapor content of the second gas;
compare the water vapor content to a predetermined value; and
stop the extraction when the water vapor content is less than the predetermined value.

20. The terpene extraction apparatus of claim 19 wherein the terpene extraction apparatus is designed to decrease the water vapor content of the second gas as the terpene extraction apparatus proceeds.

21. The terpene extraction apparatus of claim 19 wherein the terpene extraction apparatus is designed to cause at least some dissolved terpene in the second gas to precipitate as a liquid or solid.

22. The terpene extraction apparatus of claim 19 wherein the biomass is primarily plant mass.

23. The terpene extraction apparatus of claim 22 wherein the biomass comprises one or more plants from the Cannabaceae family of plants.

24. The terpene extraction apparatus of claim 19 wherein:
the cold trap assembly has a second gas flow path between an inlet and an outlet;
the flow path between the inlet and the outlet has an interior surface and an exterior surface; and
a coolant cools the second gas via the interior surface and the exterior surface.

25. The terpene extraction apparatus of claim 24 wherein:
a first coolant cools the second gas via the interior surface; and
a second coolant cools the second gas via the exterior surface.

26. The terpene extraction apparatus of claim 24 further comprising:
a first thermowell adapted to provide temperature data representative of the interior surface; and
a second thermowell adapted to provide temperature data representative of the exterior surface.

27. The terpene extraction process of claim 19 further comprising:
a hygrometer for use in measuring the vapor content of the second gas.
